(19) United States
(12) Patent Application Publication
(10) Pub. No.: US 2013/0261194 A1
(43) Pub. Date: Oct. 3, 2013

(54) Functionalized Water-Soluble Polyphosphazenes and Uses Thereof as Modifiers of Biological Agents

(71) Applicant: Alexander K. Andrianov, Belmont, MA (US)

(72) Inventor: Alexander K. Andrianov, Belmont, MA (US)

(21) Appl. No.: 13/787,994

(22) Filed: Mar. 7, 2013

Related U.S. Application Data
(63) Continuation of application No. 13/066,785, filed on Apr. 25, 2011, now abandoned.
Application No. 13/066,785 is a continuation of application No. 12/799,449, filed on Apr. 26, 2010, now abandoned.
Application No. 12/799,449 is a continuation-in-part of application No. 11/103,000, filed on Apr. 11, 2005, now abandoned.

(51) Int. Cl. A61K 47/34

(52) U.S. Cl. CPC A61K 47/34
USPC 514/772.3; 528/321; 528/399; 527/207; 527/310

(57) ABSTRACT

A polyphosphazene polymer which includes hydrophilic side groups and interacting side groups which are capable of bonding with a biological agent of interest. The bonding may be by non-covalent bonding.

This invention relates to polyphosphazene polymers. More particularly, this invention relates to polyphosphazene polymers which include hydrophilic side groups and interacting side groups, wherein the interacting side groups are capable of bonding with a molecule of interest, preferably by non-covalent bonding. The molecule of interest may be a biological agent.

Many biological agents, including therapeutic proteins, are cleared rapidly from the circulation and, as a consequence, exhibit relatively short-lived pharmacological activity.

The systematic introduction of relatively large quantities of proteins, particularly those foreign to the human system, can give rise to undesirable side effects, such as immunogenic reactions. For other biological agents, solubility and aggregation problems have also hindered their optimal formulations It has been shown that the clearance time of such therapeutic agents can, in many cases, be increased by the covalent attachment of water-soluble polymers, such as polyethylene glycol, dextran, polyvinyl alcohol, and polyvinyl pyrrolidone.

Polyethylene glycol (PEG) is the most popular polymer choice for use in covalent modification of biologically active agents (PEGylation). Some of the most desirable characteristics of PEG are its solubility in water, lack of toxicity, and lack of immunogenicity. In its most common form PEG is a linear polymer terminated at each end with hydroxyl groups. Typically the degree of polymerization ranges from approximately 10 to approximately 2000. PEG useful for biological applications is methoxy-PEG—OH, or mPEG, in which one terminus is the relatively inert methoxy group, while the other terminus is a hydroxyl group that is subject to chemical modification. In order to couple PEG to a molecule such as a protein it is necessary to use an activated derivative” of the PEG having a functional group at the terminus suitable for reacting with some group on the protein (such as an amino group). Various derivatives have been synthesized that have an active moiety for the covalent attachment to biologically active agent. The most common method involves activating the hydroxy group on the PEG with a functionality susceptible to nucleophilic attack by the nitrogen of amino groups on the protein. The composition of the resultant graft macromolecular system is dependent on the number of available attachment sites on the protein (polypeptide or carbohydrate), the reactivity of the PEG reagent, the excess of such a reagent and the reaction conditions.

PEGylation is proven to enhance physical and chemical stability and reduce immunogenicity and antigenicity of therapeutic proteins, to increase solubility of hydrophobic drugs in water, and to eliminate aggregation of peptides and proteins. For example, Davis et al. in U.S. Pat. No. 4,179,337 have shown that proteins coupled to PEG have enhanced blood circulation lifetime because of reduced rate of kidney clearance and reduced immunogenicity. Another benefit associated with PEGylation is that water solubility is increased as a result of the high water solubility of polyethylene glycol. The increased water solubility can improve the protein's formulation characteristics at physiological pH and can decrease complications associated with aggregation of low solubility proteins. In summary, PEG modification is known to improve the clinical usefulness of the therapeutic drugs, such as proteins.

PEGylated proteins vary in the extent to which plasma circulation half life is increased, immunogenicity is reduced, water solubility is enhanced, and activity is improved. Factors responsible for these variations are numerous and include the degree to which the protein is substituted with polyethylene glycol, the chemistries used to attach the polyethylene glycol to the protein, and the locations of the polyethylene glycol sites on the protein.

Despite positive reports on PEGylated biologically active agents, their synthesis still presents certain challenges for their commercial manufacturing. Preparation of monofunctional PEG with controlled molecular weight, chemical activation of PEG, a reaction of covalent conjugation, purification of the synthesized product require sophisticated technologies and equipment, multiple step processes and dictate high development and manufacturing costs.

In addition, it has been shown that covalent binding to a synthetic water soluble polymer does not predictably enhance the biological activity of biomolecules. Some of the functional groups that have been used to activate PEG can result in toxic or otherwise undesirable residues when used for in vivo drug delivery. Covalent attachment methods can result in loss of biological activity due to the nonspecific and random attachment of multiple PEG. Conjugation of other water-soluble polymers can also lead to the loss of activity or increased immunogenicity of the complex.

For example, there exist problems associated with loss of activity and production of heterogeneous mixtures of partially modified proteins, and the use of toxic reagents during the PEGylation reaction (U.S. Pat. No. 5,766,897). Toxic reagents, such as cyanuric chloride pose a serious technical difficulty in the preparation of pharmaceutical compositions, and require numerous additional steps to ensure the purity of the final pharmaceutical preparation (U.S. Pat. No. 5,766,897).

Water-soluble complexes of polymers with proteins formed through non-covalent interactions are known. The advantages of these complexes are that they can be prepared simply by mixing the components in aqueous solutions and require no toxic chemicals or expensive purification steps. Specifically, polyphosphazene polyelectrolytes form complexes with proteins; however, they also show high antigenicity. (Andrianov et al. U.S. Pat. No. 5,494,673: Andrianov, A. K., “Design and Synthesis of Functionalized Polyphosphazencs with Immune Modulating Activity,” American Chemical Society PMSE Preprints, Vol. 88, (2003)).

There is a clear need for creating complexes wherein biologically active agents are linked to a biologically inert polymer through non-covalent links and which do not show high immunogenicity. Such method will be cost effective, simple from the manufacturing perspective, will not require knowledge of the amino acid residues essential for biological activity, and will use no toxic chemicals.

In accordance with an aspect of the present invention, there is provided a polyphosphazene polymer having the following structural formula:

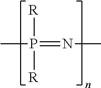

Each R in each monomeric unit of the polymer is the same or different. At least a portion of the R groups of said polymer are hydrophilic side groups R₁, and at least a portion of the R groups of the polymer are interacting side groups R₂. R₂ is capable of bonding with a molecule of interest. n is an integer from about 10 to about 300,000, preferably from about 10,000 to about 300,000.

In one embodiment, at least 80 mole % of the R groups are R₁ groups.

In one embodiment, R₁ has a structural formula selected from the group consisting of:

-A−(B₁)ₓ—(C₁)_{y}—(B₂)_{z}; and

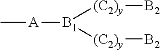

A is oxygen or nitrogen. Each of B₁ and B₂ is a substituted or unsubstituted alkyl, aryl, or alkylaryl group having from 1 to 20 carbon atoms, and each of B₁ and B₂ is the same or different. C₁ is selected from the group consisting of alkyleneoxy and substituted and unsubstituted heterocyclic groups. C₂ is alkyleneoxy. x is 0 or 1, y is from 1 to 200, and z is 0 or 1.

In one embodiment, R₁ has the formula:

-A−(B₁)ₓ—(C₁)_{y}—(B₂)_{z}, and

C₁ is alkyleneoxy. In one embodiment, A is oxygen.

In another embodiment, A is nitrogen.

In one embodiment, C₁ is methyleneoxy. In another embodiment, C₁ is ethyleneoxy. In yet another embodiment, x is 0 and z is 1. In a further embodiment, x is 1 and z is 1.

Thus, particularly preferred hydrophilic groups have the following structural formulae:

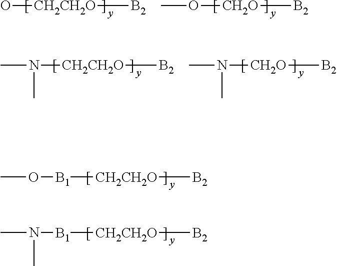

In a most preferred embodiment, A is oxygen, x is 0, C₁ is ethyleneoxy, B₂ is methyl and the hydrophilic group has the following structural formula:

—OCH₂ CH₂ OCH₂ CH₂ O3 CH₃

In another embodiment, R₁ has the structural formula:

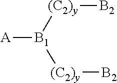

In one embodiment, A is oxygen. In another embodiment, A is nitrogen.

In yet another embodiment, C₂ is ethyleneoxy.

Thus, particularly preferred hydrophilic groups have the following structural formulae:

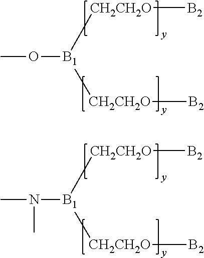

In another embodiment, R₁ has the formula: -A−(B₁)ₓ—(C₁)_{y}—(B₂)_{z}, and C₁ is a substituted or unsubstituted heterocyclic group. In one embodiment, A is oxygen, and in another embodiment, A is nitrogen.

In one embodiment, C₁ has the following structural formula:

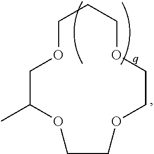

wherein q is from 1 to 5.

Particularly preferred hydrophilic groups have the following structural formulae:

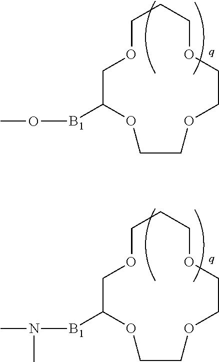

In another embodiment, C₁ is a pyrrolidone, which has the following structural

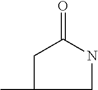

formula:

Particularly preferred hydrophilic groups have the following structural formulae:

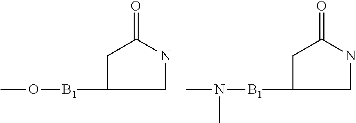

In another embodiment, the interacting side group R₂ includes (i) an ionic moiety or an ionizable moiety, (ii) a hydrophobic moiety, or (iii) a hydrogen bond forming moiety,

In one embodiment, R₂ includes an ionic moiety or an ionizable moiety. In a preferred embodiment, when R₂ includes an ionic or ionizable moiety, R₂ has the following structural formula:

—X—(Y)ₘ—Z

X is oxygen or nitrogen, Y is alkyl, aryl, alkylaryl, or alkyleneoxy. Z is an ionic or ionizable moiety, and m is from 1 to 50. Ionic and ionizable moieties which may be included in the polymer include, but are not limited to, carboxylic acid moieties, sulfonic acid moieties, sulfate moieties, amino moieties, and salts thereof.

In one preferred embodiment, the ionic moiety or ionizable moiety is a carboxylic acid moiety.

Particularly preferred R₂ groups which include a carboxylic acid moiety have the following structural formula:

—X—(Y)ₘ—COOH

X is oxygen or nitrogen. Y is alkyl, aryl, alkylaryl, or alkyleneoxy, and m is from 1 to 50. In one embodiment. X is oxygen, and in another embodiment, X is nitrogen.

In a most preferred embodiment, X is oxygen. Y is phenyl, m is 1, and the R₂ group has the following structural formula:

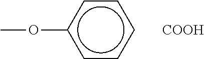

In another preferred embodiment, the ionic or ionizable moiety is a sulfonic acid moiety.

Particularly preferred R₂ groups which include a sulfonic acid moiety have the following structural formula:

X—(Y)ₘ'SO₃H

X is oxygen or nitrogen, Y is alkyl, aryl, alkylaryl, or alkyleneoxy, and m is from 1 to 50. In one embodiment, X is oxygen, and in another embodiment, X is nitrogen.

In a most preferred embodiment, X is oxygen, Y is phenyl, m is 1, and the R₂ group has the following structural formula:

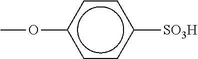

In yet another embodiment, the ionic or ionizable moiety is an amino moiety.

Particularly preferred R₂ groups which include an amino moiety have the following structural formula:

—X—(Y)ₘ—N(M)ₒ (Q)ₚ

X is oxygen or nitrogen, Y is alkyl, aryl, aralkyl, or alkyleneoxy. M is hydrogen or alkyl, and each M is the same or different. Q is a halogen. m is from 1 to 50. o is 2 or 3. p is 0 or 1.

In one embodiment, X is oxygen. In another embodiment, X is nitrogen.

In another embodiment, Y is alkoxy, and most preferably Y is ethyleneoxy.

In a further embodiment, Y is alkyl.

In yet another embodiment, p is 0.

In another embodiment, p is 1.

Particularly preferred examples of R₂ groups which include amino moieties include the following:

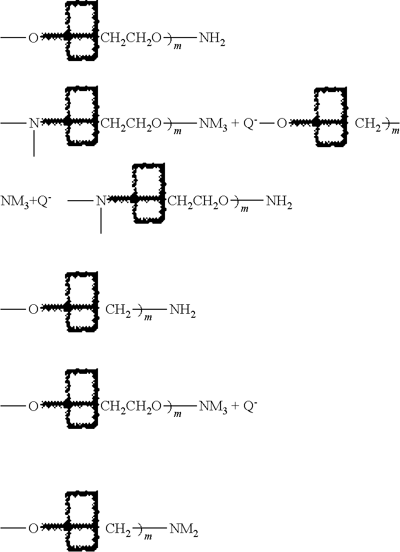

Most preferred examples of R₂ groups which include amino moieties are: —O [symbol] CH₂CH₂O—)ₘ N(CH3)₂; and —N—CH₂CH₂CH₂NH(CH₃)₂

In a further embodiment, at least a portion of the R groups are biodegradable side groups R₃. Suitable biodegradable side groups include, but are not limited to, chlorine, amino acids, amino acid esters, and imidazolyl, glycinyl, glyceryl, glucosyl, and ethoxy groups.

In yet another embodiment, at least a portion of the R groups are targeting side groups R₄. Targeting side groups which may be employed include, but are not limited to, antibodies, lectins, tri- and tetraantennary glycosides, transferrin, and other molecules which are bound specifically by receptors on the surfaces of cells of a particular type.

The polyphosphazene polymers of the present invention may be prepared by a macromolecular nucleophilic substitution reaction of a polyphosphazene substrate, such as poly(dichlorophosphazene), with a wide range of chemical reagents or mixture of reagents in accordance with methods known to those skilled in the art. Preferably, the polyphosphazene polymers of the present invention are made by reacting poly(dichlorophosphazene) with an appropriate nucleophile or nucleophiles that displace chlorine. Desired proportions of R₁ and R₂ groups, as well as R₃ and R₄ groups if needed, can be obtained by adjusting the quantities of the corresponding nucleophiles that are reacted with poly(dichlorophosphazene) and the reaction conditions if necessary.

Alternatively, the polyphosphazene substrate is a polydicholorophosphazene derivative wherein some of the chlorine atoms have been replaced with organic side groups. Thus, the substrate is a copolymer of polydichlorophosphazene and polyorganophosphazene.

The nucleophilic substitution reaction of the polyphosphazene substrate with the desired proportions of the R₁ and R₂ groups, and R₃ and R₄ groups if needed, takes place in an appropriate organic solvent. Organic solvents in which the reaction is effected include, but are not limited to, diglyme, chlorobenzene, dichlorobenzene, dichloroethane, N,N-dimethylfoamide (DMF), N,N-dimethylacetamide, dioxane, tetrahydrofuran (THF), toluene, methylsulfoxide, and dimethylsulfone, and mixtures thereof. The reaction mixture then is subjected to appropriate reaction conditions, including heating, cooling, and/or agitation. The reaction mixture then may be filtered, if necessary, and organic and aqueous layers then are separated. Depending on the polymer structure, the polymer is isolated from the aqueous or organic phase by precipitation. The resulting polymer then is dried. The organic solvent and reaction conditions employed are dependent upon a variety of factors, including, but not limited to, the polyphosphazene substrate employed, the R₁ and R₂ groups, and R₃ and R₄ groups, if included, and the proportions thereof.

Preferably, the polyphosphazene polymers of the present invention have a molecular weight of from about 1,000 g/mole to about 10,000,000 g/mole, preferably from about 20,000 g/mole to about 800,000 g/mole.

In one embodiment, the R₁ and R₂ groups, and R₃ and R₄ groups, if employed, are distributed randomly throughout the polymer.

Thus, with the proviso that the polymer includes both R₁ and R₂ groups, each monomeric unit of the polymer may be any one of the following:

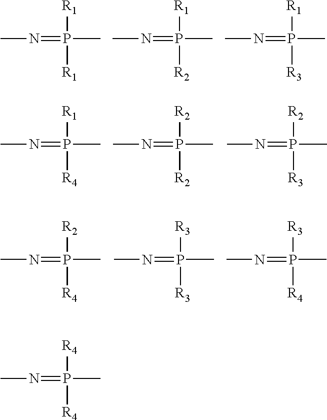

These monomeric units may be distributed randomly or in blocks throughout the polymer, provided that the polyphosphazene polymer includes both R₁ and R2 groups.

Furthermore, in accordance with the present invention, the polyphosphazene polymer may include more than one specific R₁ group, and/or may include more than one specific R2 group, and/or may include more than one specific R₃ group, and/or may include more than one specific R₄ group.

In a preferred embodiment, the polyphosphazcne copolymer forms a complex with a biological agent and preferably such complex is formed through non-covalent interactions or bonds. The term “non-covalent interactions” refers to intermolecular interaction among two or more separate molecules which does not involve a covalent bond. Intermolecular interaction is dependent upon a variety of factors, including, for example, the polarity of the involved molecules, and the charge (positive or negative), if any, of the involved molecules. Non-covalent associations are selected from ionic or electrostatic interactions, hydrophobic interactions, hydrogen bonding, dipole-dipole interactions, van der Waals forces, and combinations thereof.

Alternatively, the polyphosphazene copolymer can form water-soluble complexes through the establishment of at least two covalent bonds with a biological agent.

Water-soluble macromolecular complexes can be prepared to contain one molecule of biologically active agent per one molecule of polyphosphazene copolymer. Alternatively, the polyphosphazene copolymer can be linked to two or more bioactive molecules. In general, the molar ratio of polyphosphazene polymer to the one or more biologically active agent(s) is from about 1:10 to about 10:1, preferably at about 1:1. In certain cases, the administration of multimeric complexes that contain more than one biologically active polypeptide or drug leads to synergistic benefits. A complex containing two or more identical biomolecules may have substantially increased affinity for the ligand or active site to which it binds relative to the monomeric biomolecule. In addition to a bioactive agent, a complex can contain a molecule or functional group (i.e., targeting moiety, R₄) that can direct the complex to the ligand or active site.

Biological agents with which the polymers of the present invention may be complexed include, but not limited to, water-soluble molecules possessing pharmacological activity, such as a peptide, protein, enzyme, enzyme inhibitor, antigen, cytostatic agent, anti-inflammatory agent, antibiotic, DNA construct, RNA construct, or growth factor. Examples of therapeutic proteins are interleukins, albumins, growth hormones, aspariginase, superoxide dismutase, and monoclonal antibodies. Biological agents include also water-insoluble drugs, such as camptothecin and related topoisomerase I inhibitors, gemcitabine, taxanes, and paclitaxel derivatives. Other useful compounds include, for example, certain low molecular weight biologically active peptides, including peptidoglycans, as well as other anti-tumor agents; cardiovascular agents such as forskolin; anti-neoplastics such as combretastatin, vinblastine, doxorubicin, mytansine; anti-infectives such as vancomycin, erythromycin; anti-fungals such as nystatin, amphotericin B, triazoles, papulocandins, pneumocandins, echinocandins, polyoxins, nikkomycins, pradimieins, benanomicins; anti-anxiety agents, gastrointestinal agents, central nervous system-activating agents, analgesics, fertility agents, anti-inflammatory agents, steroidal agents, anti-urecemic agents, cardiovascular agents, vasodilating agents, vasoconstricting agents and the like.

The biological agents may be in a variety of physical states, including solid, liquid, solution, or suspension, and such agents may, be encapsulated in biodegradable or hydrogel microspheres, microcapsules and nanospheres, or liposomes.

Complexes of polyphosphazene polymers of the present invention may be prepared in aqueous solutions. The addition of aqueous solutions of biological agents, such as proteins, to the polyphosphazene polymers leads to the formation of macromolecular complexes. Complex formation may be conducted at a wide range of temperatures, and preferably from about 0° C. to about 40° C. The addition of the protein solution to the polyphosphazene polymers may be conducted with mechanical stirring or vortex. Various concentrations of polyphosphazene polymer and biological agent may be employed. Preferably, such concentrations are from about 0.01% wt./wt. to about 2% wt./wt. In some instances, a mediating compound may be added to the polyphosphazene polymer and biological agent in order to facilitate interactions between the polyphosphazene polymer and biological agent. Mediating compounds which may be employed include, but are not limited to, polyamines, such as spermine, for example. The polyphosphazene polymer, biological agent, and mediating compound, if needed, are allowed to stand for a period of time sufficient to form macromolecular complexes of the polyphosphazene polymers and biological agents. Reaction times vary, depending upon the specific polyphosphazene polymers and biological agents employed; typically, reaction times are in the order of from about 1 minute to about 360 minutes. The sizes of the resulting complexes are dependent upon a variety of factors, such as pH, temperature, and the specific polyphosphazene polymer employed.

The resulting complexes of the present invention have improved properties, including but not limited to, increased solubility, increased stability, extended half-lives, increased potency, and reduced antigenicity and immunogenicity.

The invention now will be described with respect to the following examples: however, the scope of the present invention is not intended to be limited thereby.

### EXAMPLE 1

### Synthesis of poly[(methoxyethoxyethoxy)(decoxy)(carboxylatophenylamino)phosphazene]

0.058 g (0.30 mmol) of butyl 4-aminobenzoate in 2.25 mL of diglyme was added to 0.116 g (1 mmol) of polydichlorophosphazene (PDCP) in 15 ml of diglyme at 50° C. under stirring. The temperature was increased to 70° C. and the reaction was continued for 10 minutes while stirring. 2.25 mL of sodium decanoxide solution (0.15 mmol), prepared by reacting 0.65 g of n-decanol (4.05 mmol) with 0.1 g of sodium hydride (3.96 mmol) in 60 mL of tetrahydrofuran (THF), was added to the reaction mixture. The reaction was continued for 1 hour at 70° C. Then, 1.36 mL (2.1 mmol) of sodium salt of di(ethylene glycol)methyl ether solution, prepared by reacting 8.16 g ether (68 mmol) of di(ethylene glycol) methyl with 1.2 g (47.5 mmol) of sodium hydride in 20.6 mL diglyme, was added to the reaction mixture.

Temperature was increased to 110° C. and the reaction mixture was stirred for 2 hours at 110° C. Then the reaction mixture was cooled down to 90° C. 1 mL of 12.7 N aqueous potassium hydroxide was added to the mixture and the reaction was continued for 1 hour at 90° C. while stirring. The polymer was recovered by precipitating with a mixture of 75 mL of THF and 5 mL of 4 N aqueous hydrochloric acid. The polymer was dried overnight at room temperature. Then the precipitate was re-dissolved in 5 mL of distilled water, and pH of the solution was adjusted to pH 7.5-8.5 using 5% aqueous potassium hydroxide. The polymer was purified using size-exclusion reparative chromatography and lyophilized.

### EXAMPLE 2

### Synthesis of poly[(methoxyethoxyethoxy)(decoxy)(dimethylaminoethoxy)phosphazene]

A solution of sodium decanoxide was prepared by reacting 1.896 g (12 mmol) of n-decanol with 0.253 g (10 mmol) of sodium hydride in 27.8 mL of THF. 0.30 mL of this solution (0.10 mmol of sodium decanoxide) was added to 0.116 g (1 mmol) of PDCP solution in 15 mL of diglyme at 50° C. while stirring. The temperature was increased to 70° C. and reaction continued for another hour. A solution of sodium salt of di(ethylene glycol)methyl ether was prepared by reacting 4.9 g (40.8 mmol) of di(ethylene glycol)methyl ether with 0.859 g (34 mmol) sodium hydride in 24.2 mL of diglyme. 1.5 mL of this solution (1.7 mmol of di(ethylene glycol)methyl ether) was added to the reaction mixture. The temperature then was increased to 90° C. and reaction continued for two hours. The temperature was then decreased to 55° C. A solution of sodium salt of 2-(2-dimethylaminoethoxy)ethanol was prepared by reacting 2.56 g (19.2 mmol) of 2-(2-dimethylaminoethoxy)ethanol with 0.404 g (15.8 mmol) sodium hydride in 17 mL diglyme. 1 mL of this solution (0.8 mmol of amine) was added to the reaction mixture. The reaction was continued for 22 hours at 55° C. Then the temperature was decreased to ambient. The polymer was recovered by precipitating with a mixture of 75 mL of THF and 5 mL of 4 N aqueous hydrochloric acid. The polymer was dried overnight at room temperature. Then, the precipitate was re-dissolved in 5 mL of deionized water, and the pH of the solution was adjusted to pH 7.5-8.5 using 5% aqueous potassium hydroxide. The polymer was purified using size-exclusion reparative chromatography and lyophilized.

The disclosures of all patents and publications, including published patent applications, hereby are incorporated by reference to the same extent as if each patent and publication specifically and individually were incorporated by reference.

It is to be understood, however, that the scope of the present invention is not to be limited to the specific embodiments described above. The invention may be practiced other than as particularly described and still be within the scope of the accompanying claims.

What is claimed is:

1. A polyphosphazene polymer having the following structural formula: 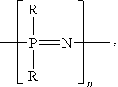
wherein each R in each monomeric unit of said polymer is the same or different, and wherein at least a portion of the R groups of said polymer are hydrophilic side groups R₁, and at least a portion of the R groups of said polymer are interacting side groups R₂, wherein R₂ is capable of bonding with a molecule of interest, and n is an integer from about 10 to about 300,000.

2. The polymer of claim 1 wherein R₁ has a structural formula selected from the group consisting of:
-A−(B ₁)ₓ—(C₁)_{y}—(B₂)_{z}; and 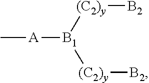
wherein A is oxygen or nitrogen, each of B₁ and B₂ is a substituted or unsubstituted aryl, or alkylaryl group having from 1 to 20 carbon atoms, and each of B₁ and B₂ is the same or different, C₁ is selected from the group consisting of alkyleneoxy and substituted and unsubstituted heterocyclic groups, C₂ is alkyleneoxy, x is 0 or 1,y is from 1 to 200, and z is 0 or 1.

3. The polymer of claim 2 wherein R₁ has the formula:
-A−(B₁)x—(C₁)_{y}—(B₂)_{z}, and C₁ is alkyleneoxy.

4. The polymer of claim 3 wherein A is nitrogen.

5. The polymer of claim 3 wherein A is oxygen.

6. The polymer of claim 3 wherein C₁ is methyleneoxy.

7. The polymer of claim 3 wherein C₁ is ethyleneoxy.

8. The polymer of claim 3 wherein x is 0 and z is 1.

9. The polymer of claim 3 wherein x is 1 and z is 1.

10. The polymer of claim 2 wherein R₁ has the formula:
-A−(B ₁)x−(C₁)y—(B₂)z, and C₁ is a substituted or unsubstituted heterocyclic group.

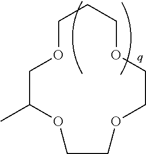

11. The polymer of claim 10 wherein C₁ has the following structural formula:
, wherein q is from 1 to 5.

12. The polymer of claim 10 wherein x is 1 and z is 0.

13. The polymer of claim 10 wherein C₁ has the following structural formula:

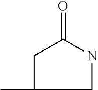

14. The polymer of claim 10 wherein A is oxygen.

15. The polymer of claim 10 wherein A is nitrogen.

16. The polymer of claim 1 wherein R₂ includes (i) an ionic moiety or an ionizable moiety, (ii) a hydrophobic moiety, or (iii) a hydrogen bond forming moiety.

17. The polymer of claim 16 wherein R₂ includes an ionic moiety or an ionizable moiety.

18. The polymer of claim 17 wherein said ionic moiety or ionizable moiety is selected from the group consisting of carboxylic acid moieties, sulfonic acid moieties, sulfate moieties, amino moieties and salts thereof.

19. The polymer of claim 18 wherein said ionic moiety or ionizable moiety is a carboxylic acid moiety.

20. The polymer of claim 18 wherein said ionic moiety or ionizable moiety is a sulfonic acid moiety.

21. The polymer of claim 18 wherein said ionic moiety or ionizable moiety is an amino moiety.

22. The polymer of claim 1 wherein at least a portion of said R groups are biodegradable side groups R₃.

23. The polymer of claim 22 wherein said biodegradable side groups R₃ are selected from the group consisting of chlorine, amino acids, amino acid esters, imidazolyl groups, glycinyl groups, glyceryl groups, glucosyl groups, and ethoxy groups.

24. The polymer of claim 1 wherein at least a portion of said R groups are targeting side groups R₄.

25. The polymer of claim 24 wherein said targeting side groups R₄ are selected from the group consisting of antibodies, lectins, triantennary glycosides, tetraantennary glycosides and transferrin.

26. A composition, comprising:
the polyphosphazene polymer of claim 1; and
at least one biological agent of interest.

27. The composition of claim 26 wherein the molar ratio of said polyphosphazene polymer to said at least one biological agent in said composition is from about 1:10 to about 10:1.

28. The composition of claim 27 wherein the molar ratio said polyphosphazene polymer to said at least one biological agent in said composition is about 1:1.

29. The polymer of claim 1 wherein at least 80 mole % of the R groups are R₁ groups.
